(19) United States
(12) Patent Application Publication
(10) Pub. No.: US 2014/0141032 A1
(43) Pub. Date: May 22, 2014

(54) TEMPO-mediated glycoconjugation of immunogenic composition against Campylobacter jejuni with improved structural integrity and immunogenicity

(71) Applicants: Patricia Guerry, Silver Spring, MD (US); Mario Artur Monteiro, Guelph (CA)

(72) Inventors: Patricia Guerry, Silver Spring, MD (US); Mario Artur Monteiro, Guelph (CA)

(21) Appl. No.: 13/681,773

(22) Filed: Nov. 20, 2012

(51) Int. Cl. A61K 39/385; A61K 47/48

(52) U.S. Cl. CPC A61K 39/385; A61K 47/4833
USPC 424/194.1; 530/411; 530/409

(57) ABSTRACT

Immunogenic capsule polysaccharide polymer composition, and its method of producing, with improved structural integrity and immunogenic properties. The invention also relates to a method of using the compositions to elicit an immune response to Campylobacter jejuni.

### CROSS-REFERENCES TO RELATED APPLICATIONS

This application claims the benefit of U.S. Provisional Application, No. 61/629,823, filed 23 Nov. 2011.

### BACKGROUND OF INVENTION

1. Field of Invention

The inventive subject matter relates to a Campylobacter polysaccharide-protein conjugate with improved structural integrity and immunochemical properties and a method of producing said polysaccharide protein conjugate and use to stimulate anti-Campylobacter immunity.

2. Background Art

C. jejuni is a leading cause of diarrheal disease worldwide and a documented threat to US military personnel (Taylor, Current status and future trends. Amer. Soc. Micro., (1992); Tauxe, Current status and future trends. Amer. Soc. Micro.(1992). The symptoms of Campylobacter mediated enteritis include diarrhea, abdominal pain, and fever and often accompanied by vomiting. Stools usually contain mucus, fecal leukocytes, and blood, although watery diarrhea is also observed (Cover and B laser, Ann. Rev. Med., 40: 269-285 (1999)). However, despite the importance of this organism to human disease, there are no licensed vaccines against C. jejuni.

An interesting recent revelation regarding the Campylobacter genome sequence was the presence of a complete set of capsule transport genes similar to those seen in type II/III capsule loci in the Enterobactericeae (Parkhill et al., Nature, 403: 665-668 (2000); Karlyshev et al., Mol. Microbiol., 35: 529-541 (2000)).

Subsequent genetic studies in which site-specific mutations were made in several capsule transport genes indicated that the capsule was the serodeterminant of the Penner serotyping scheme (Karlyshev et al., Mol. Microbiol., 35: 529-541 (2000)). The Penner scheme (or HS for heat stable) is one of two major serotyping schemes of campylobacters and was originally thought to be based on lipopolysaccharide O side chains (Moran and Penner, J. Appl. Microbiol., 86: 361-377 (1999)).

Currently it is believed that the structures previously described as O side chains are, in fact, capsules. Conjugate vaccines containing bacterial polysaccharides may be an effective tool in controlling bacterial infections (Jennings, Adv. Carbohydr. Chem. Biochem., 41: 155-208 (1983); Eby, Pharm. Biotechnol., 4: 695-718 (1995); Buskas, et al, Chem. Commun., 36: 5335-5349 (2009); Wu, et al., Infect. Immun., 78: 1276-1283 (2010)).

### SUMMARY OF INVENTION

An object of this invention is an anti-C. jejuni immunogenic composition, comprising a polysaccharide conjugate with improved immunochemical properties. Induction of immune responses against Campylobacter by administration of polysaccharide polymers is advantageous due to the low likelihood of inducing Guillain-Barre syndrome (GBS).

Another object of the invention is a method of producing a polysaccharide-protein conjugate that retains structural and immunochemical integrity utilizing a method wherein 2,2,6,6-tetramethylpiperidin-1-oxyl (TEMPO) is used to stoichiometrically oxidize primary alcohols of polysaccharides. Yet, another object of the invention is a method of administering the carrier conjugated or unconjugated anti-C. jejuni capsule polysaccharide composition in order to induce an immune response.

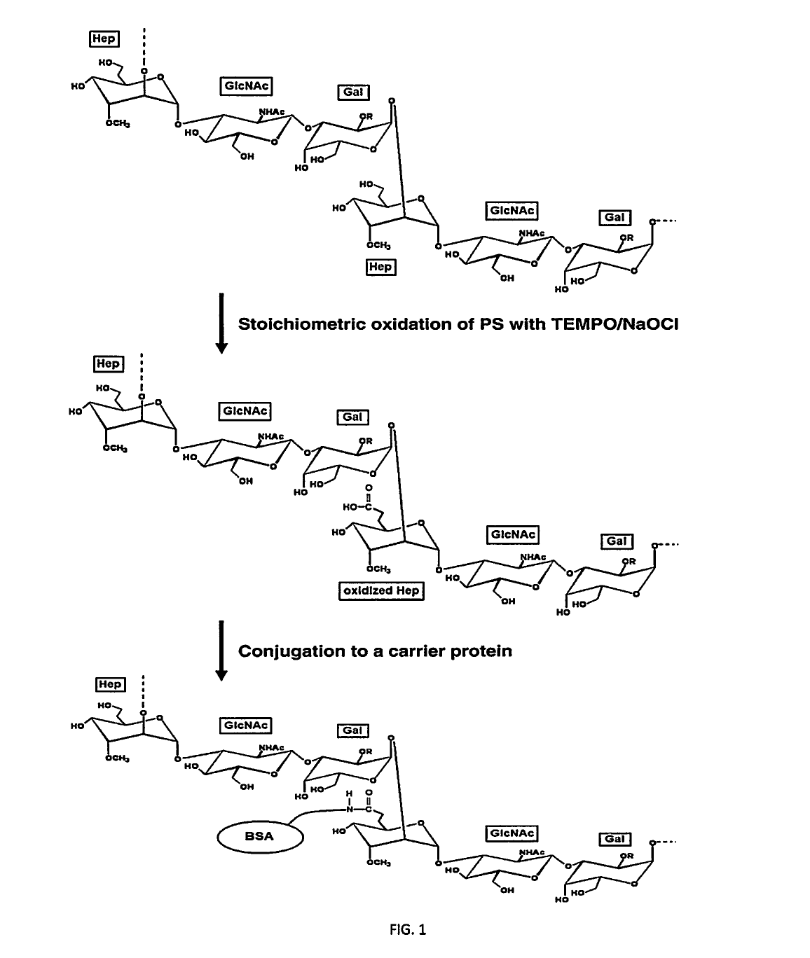

FIG. 1. Schematic representation of controlled oxidation scheme with TEMPO/NaOCl. In this example and illustration, oxidation occurs on the C-7 of the heptose unit, followed by coupling of the CPS to carrier protein through the newly oxidized carboxylic acid at C7 of the heptose residue.

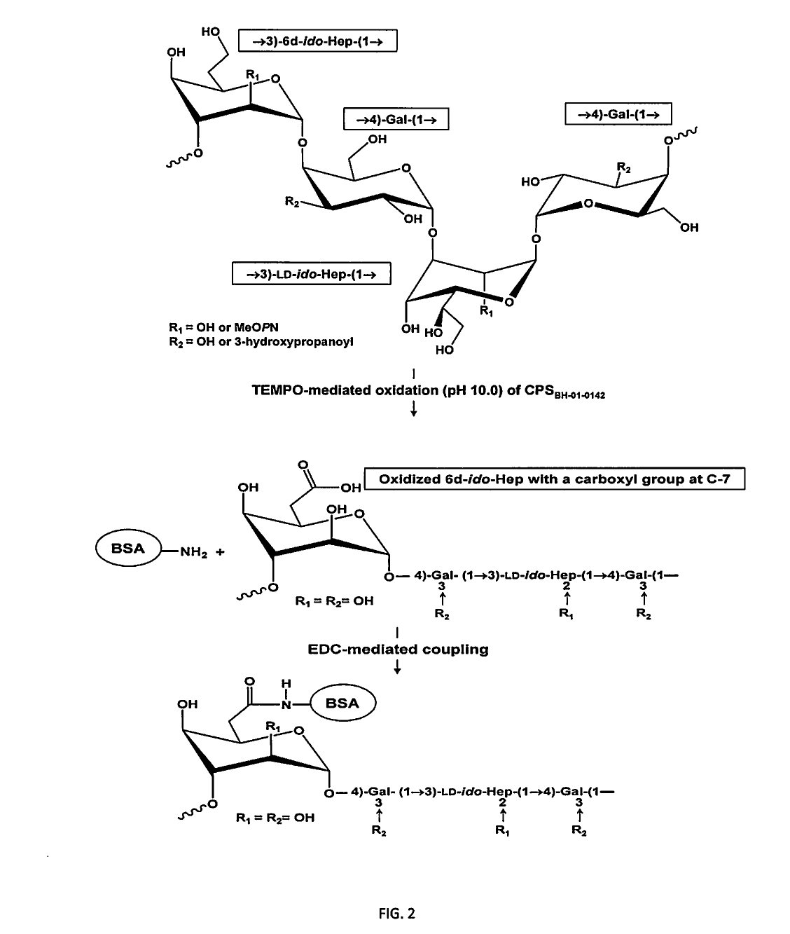

FIG. 2. Schematic representation of selective oxidation at C-7 of 6d-ido-Hep via TEMPO-mediated oxidation at pH 10.0 followed by EDC-mediate coupling. In this example coupling is of BH-01-0142 capsule polysaccharide is conjugated to BSA.

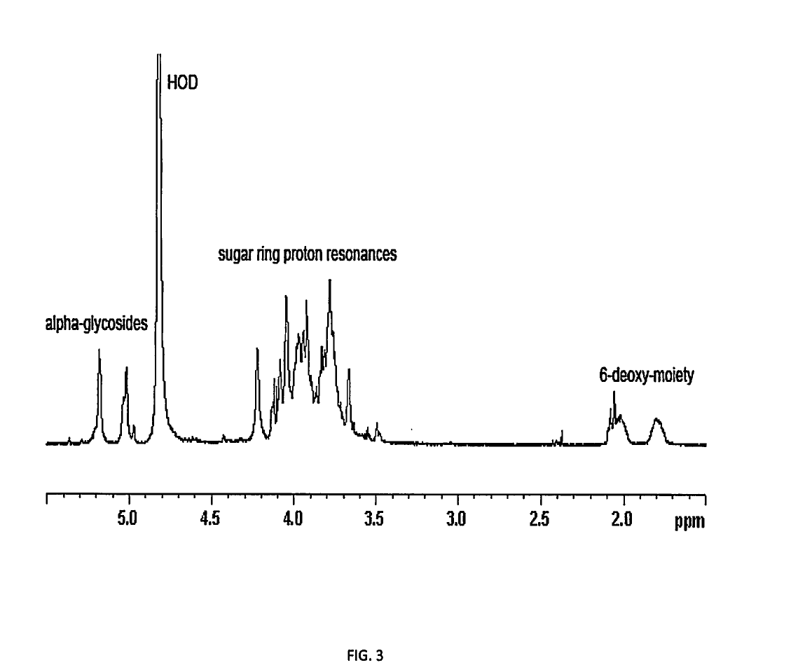

FIG. 3. 1D ^{1H} NMR spectrum of C. jejuni BH-01-0142 of the activated CPS by TEMPO oxidation at pH 10.0.

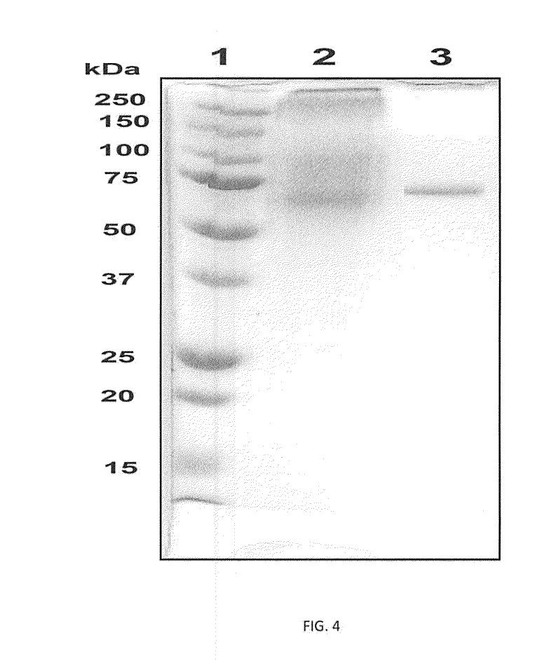

FIG. 4. Gel electrophoresis of C. jejuni BH-01-0142 conjugate.

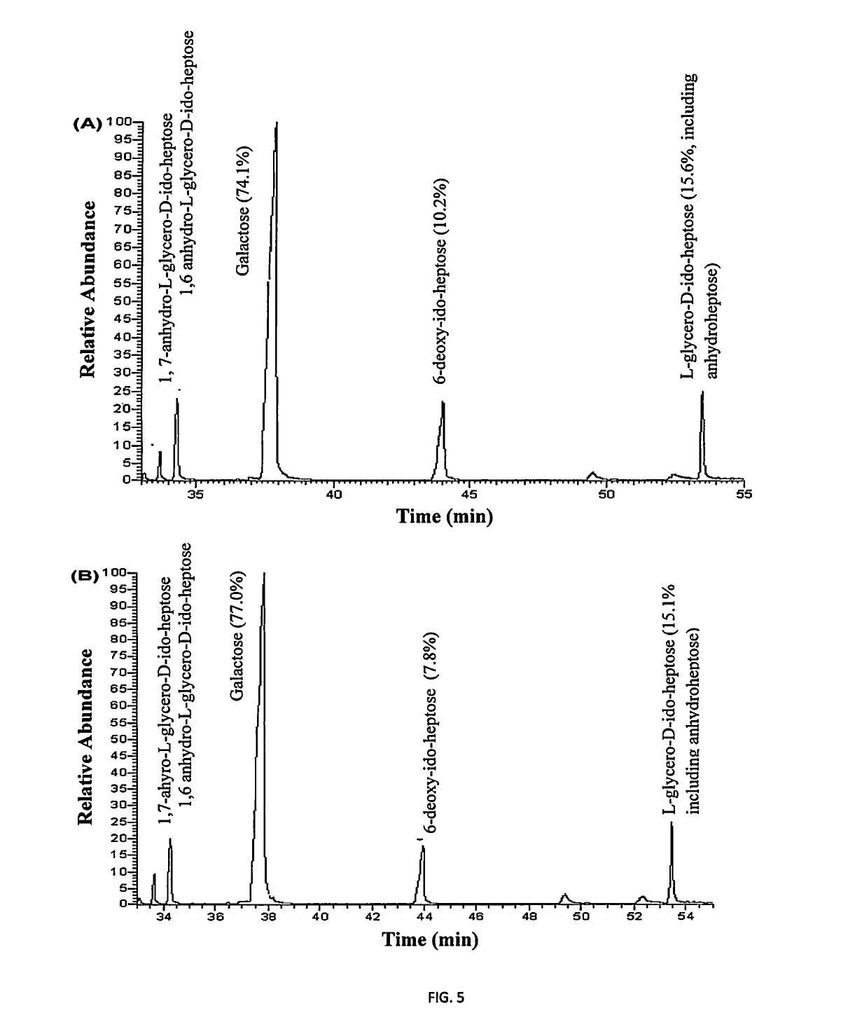

FIG. 5 GLX profile of GLC-MS of alditol acetate derivatives of (A) intact CPS; (B) the activated CPS of C. jejuni BH-01-0142 by TEMPO oxidation at pH 8.0.

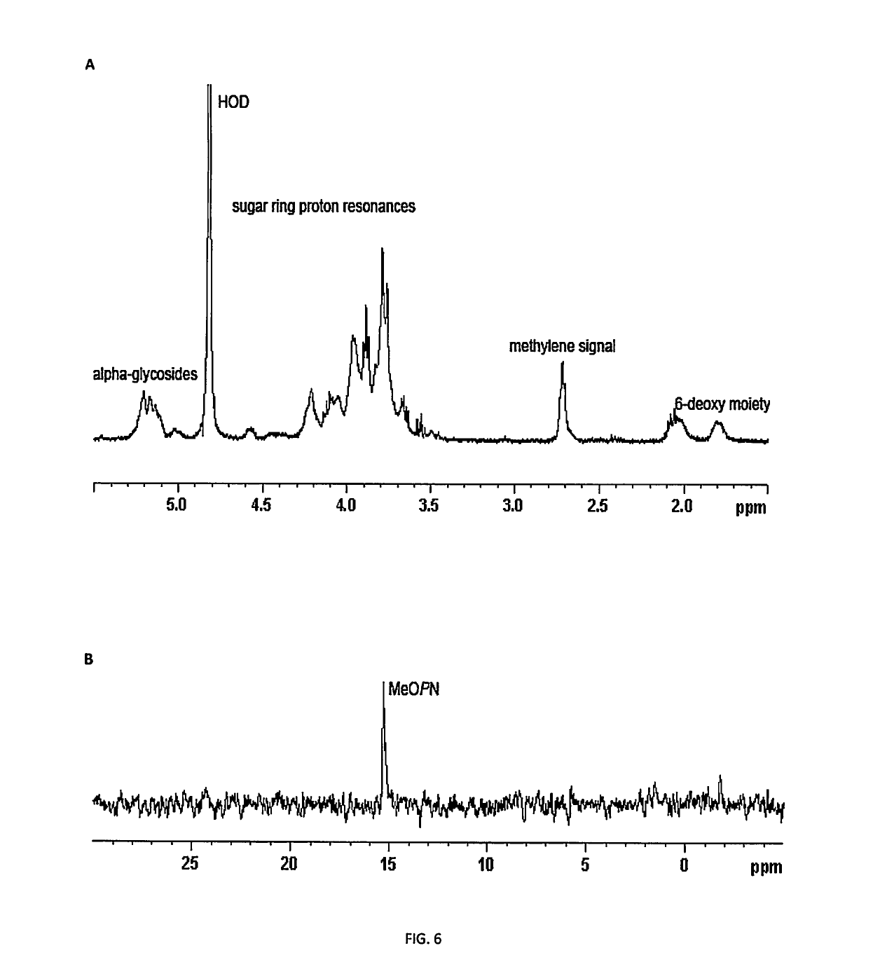

FIG. 6 (A) 1D ¹H NMR spectrum: and (B) 1D ³¹ P NMR spectrum of C. jejuni BH-01-0142 of the activated CPS by TEMPO-mediated oxidation at pH 8.0.

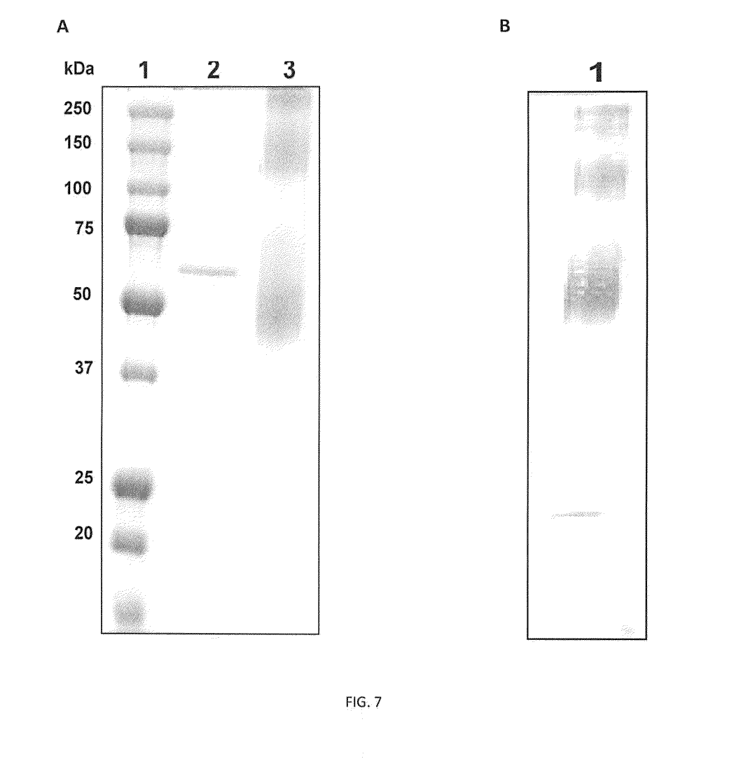

FIG. 7 SDS-PAGE electrophoresis and immunoblot of C. jejuni BH-01-0142 capsular conjugate.

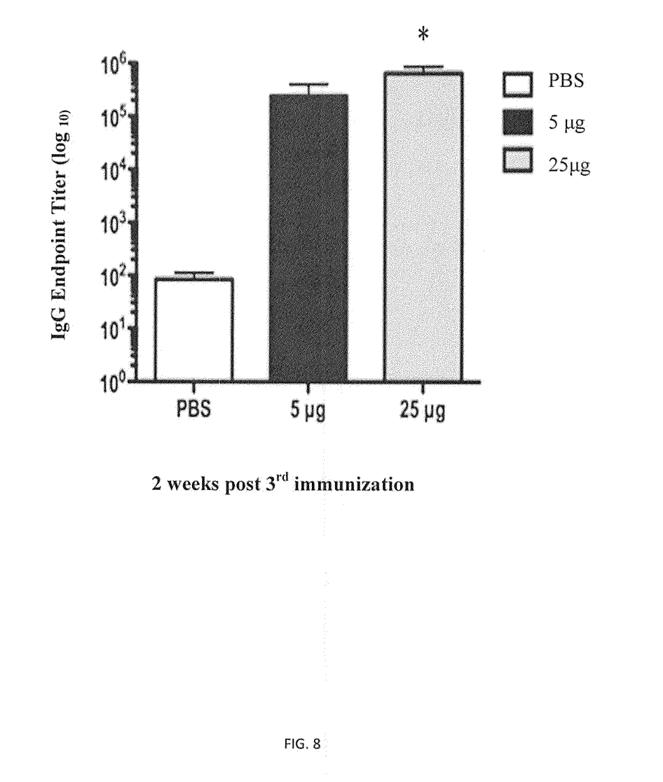

FIG. 8 IgG endpoint titer of mice immunized with BH-01-0142-CRM₁₉₇.

### DETAILED DESCRIPTION OF PREFERRED EMBODIMENTS

The chemical structures of the capsule/O side chains of several Penner serotypes of Campylobacter have been determined. These structures include several unusual sugar structures (Aspinall, et al., Carbohydrate Res. 231: 13-30 (1992); Pace, et al., U.S. Pat. No. 5,869,066; Karlyshev, et al., Mol. Micro., 55: 90-103 (2005); Carbohydrate Res. 340: 2218-2221 (2005); Chen, et al., Carbohydrate Res., 343: 1034-1040 (2008).

Avoiding disruption to the structural integrity of the polysaccharides in polysaccharide conjugates is important to maximize the immunogenicity of polysaccharide-conjugate immunogenic compositions. However, polysaccharides typically do not express functional groups that are readily available for covalent bond formation to protein carriers.

Traditional methods of polysaccharide conjugation to proteins involves modification of the polysaccharide and/or protein, often followed by the addition of a spacer, with subsequent multiple steps to join the molecules.

In a preferred embodiment, oxidation of polysaccharides is achieved utilizing 2,2,6,6-tetramethylpiperidin-1-oxyl (TEMPO) mediated oxidation (Zuchao, et al., Carbohydrate Research 346: 343-347 (2011)). TEMP selectively oxidizes primary alcohols to carboxylic acids. Therefore, in this embodiment, stoichiometric oxidation by TEMP of the capsule polysaccharides produced by Campylobacter jejuni is followed by the conjugation to protein carrier. As an example, as illustrated in FIG. 1, the C. jejuni capsule trisaccharide, composed of galactose (Gal), 3-0-methyl-6-deoxy-altro-heptose (6dHep) and N-acetyl-glucosamine (GlcNAc), non-stoichiometrically substituted at O-2 of Gal by O-methyl-phosphoramidate (MeOPN) is oxidized followed by conjugation to protein carrier.

The embodied method, however, contemplates the conjugation of other Campylobacter jejuni polysaccharides to other protein carriers. As examples, the Campylobacter jejuni polysaccharides that are envisioned to be coupled to protein include:
→4)-[P→3]alpha-D-Gal-(1→3)-[P→2/7]-6d-alpha-D-ido-Hep-(1→;
→4)-[P→3]-alpha-D-Gal-(1→3)-[P→2]-L-glycero-alpha-D-ido-Hep-(1→;
→3)-6-d-β-D-ido-Hep-(1→4)-β-D-GlcNAc-(1→; and
α-D-Gal-(1→[-2)-6d-3-O-Me-α-D-altro-Hep-(1→3)-β-D-GlcNAc-(1→3)-α-D-Gal-]ₙ.

An additional embodiment is a method of conjugating polysaccharides to carrier proteins comprising limiting the number of monosaccharides oxidized per polysaccharide chain. Typically, only 2-3 monosaccharide units in each chain are oxidized.

In other embodiments, polysaccharide capsule structures from other bacterial species are oxidized and conjugated using TEMPO-mediated glycoconjugation method. As examples, the stoichiometric oxidation by TEMPO of nigeran [(1→3)-α-glucan] and amylase [(1→4)-α-glucan], and the capsule polysaccharide produced by the Actinobacillus suis (serotype O:1; CPS_{Actinobacillus}), followed by their conjugation to BSA.

In a preferred embodiment, capsule polysaccharide is conjugated to a protein by:
a. reacting a capsule polysaccharide with 2,2,6,6-tetramethylpiperidin-1-oxyl (TEMPO);
b. exposing TEMPO reacted polysaccharide of step (a) to TEMPO and oxidant;
c. exposing oxidized polysaccharide of step (b) to a protein carrier in the presence of a coupling agent.

The oxidant can be any number of compound, however in one embodiment NaBr and/or NaOCl is utilized. Oxidant is typically exposed to limiting amounts of NaOCl. Any number of potential coupling agents can be utilized, however, as illustrated in FIG. 1, in one embodiment, 1-ethyl-3-(3-dimethylaminopropyl)carbodiimide can be utilized. Reacting to TEMPO is conducted at a pH range of 8.0 to 10.0 at a temperature of 23° C. to 37° C. In one embodiment, creation of the carboxylic acid residue is on the C-7 of 6d-ido-Hep.

### EXAMPLE 1

### Methods of Oxidation and Conjugation of Amylose and Nigeran

An embodiment of the current invention is a method of producing an immunogenic composition comprising a polysaccharide conjugate to Campylobacter with improved immunochemical properties due to improved retention of structural integrity. The embodied method comprises the TEMPO oxidation of the polysaccharide, using stochiometric amounts of TEMPO. The oxidized polysaccharide is then directly conjugated to a carrier protein using the newly created carboxylic acid units as functional groups.

Initial examination of the amounts of reagents necessary for stoichiometric oxidation of the polysaccharide was first conducted using amylose and nigeran. The intent was to develop a method of controlled oxidation. Therefore, amylose (approximately 1500 Da) and nigeran (approximately 550 Da) were oxidized by using different combinations of TEMPO-NaBr—NaClO. The results and conditions for the oxidations are illustrated in Table 1.

TABLE 1

| | | | | | | | Oxi- |
|---|---|---|---|---|---|---|---|
| | Reac- | | | | NaClO | | dizied |
| | tion | PS | TEMPO | NaBr | (4%) | Reaction | (PS) |
| PS^{a} | no. | (mg) | (mg) | (mg) | (mL) | time (hrs) | (%) |
| Amylose | 1 | 12.3 | 0.042 | 0.6 | 0.042 | 4 | 5 |
| | 2 | 24.0 | 0.168 | 2.4 | 0.168 | 4 | 5 |
| | 3 | 20.4 | 0.8 | 12.0 | 2.0 | 12 | 62 |
| | 4 | 10.5 | 0.2 | 3.0 | 0.5 | 24 | 35 |
| | 5 | 10.1 | 0.1 | 1.5 | 0.25 | 24 | 25 |
| | 6 | 10.5 | 0.1 | 1.5 | 0.125 | 24 | 15 |
| | 7 | 11.1 | 0.1 | 1.5 | 0.0625 | 24 | 10 |
| Nigeran | | 22.0 | 0.168 | 2.4 | 0.168 | 22 | 20 |
| | | 18.6 | 0.8 | 12.0 | 2.0 | 12 | 50 |
| | | 10.4 | 0.2 | 3.0 | 0.5 | 24 | 50 |
| | | 10.4 | 0.1 | 1.5 | 0.25 | 24 | 30 |
| | | 10.1 | 0.1 | 1.5 | 0.125 | 24 | 20 |
| | | 10.1 | 0.1 | 1.5 | 0.0625 | 24 | 15 |

^{a}Polysaccharide

In these studies, TEMPO, NaBr and NaClO (4%, pH was adjusted to 10) were added to a solution of polysaccharide in water (2 mL/10 mg sugar) at 0° C. The pH value of the reaction mixture was kept at 10 by continuous addition of 0.5 M NaOH. The mixture was stirred at 0° C. for about 4-24 hours until a stable pH value was achieved. The reaction was quenched by the addition of ethanol (0.1 mL/10 Mg polysaccharide) and the mixture was dialyzed against de-ionized water overnight, followed by lypholization to yield the oxidized products. The reaction conditions for each oxidation are summarized in Table. 1.

As illustrated in Table 1, under the first two conditions (reactions 1 and 2), the same percentage of sugar units in amylase was oxidized, as estimated by ¹H NMR spectroscopy, with two anomeric resonances being observed, at 5.56 ppm for the oxidized unit (now glucuronic acic; GlcA) and at 5.42 ppm for Glc residue. A longer reaction time was allowed in the third reaction, which led to 62% oxidation. The ¹³C NMR spectrum of the oxidized preparation from reaction 3 clearly showed the carboxylic acid resonances at 176 ppm. No proton or carbon resonances characteristic of aldehyde groups were observed. However, in the case that aldehydes are detected, the activated PS, or the conjugate, may be reduced with NaBH₄.

Using the data from the earlier trials, other conditions (reactions 3-7) for oxidation of amylose were designed. The results are illustrated in Table 1. It is noted that the oxidized polysaccharide from reaction 7 showed 10% of oxidized monosaccharide units. This level is highly encouraging since this level of oxidation should not be disruptive to polysaccharide structural integrity and enough carboxylic acids would be available for successful conjugation to protein. Interestingly, oxidation of another glucan, nigeran, was investigated. Under similar conditions as for amylose, the oxidation levels of nigeran were slightly higher, as illustrated in Table 1. These data suggest that hypochlorite was a key determinant in carboxylic acid formation (see reactions 5 and 6).

The oxidized polysaccharides were conjugated to protein carrier (i.e., BSA) by first dissolving the oxidized polysaccharides in MES buffer (pH 5.5, 2 mL/1.2 mg of polysaccharide) to which 1-ethyl-3-(3-dimethylaminopropyl) carbodiimide (EDC) (10 μL/1.2 mg polysaccharide) was added, and followed by the addition of BSA (0.3-14.4 mg/1.2 mg sugar). The pH value of the reaction mixture was adjusted to 5.5 by adding 0.5 M HCl. The mixture was stirred at 23° C. or 37° C. for 1-3 days, and then dialyzed against de-ionized water for 1-3 days to remove unreacted polysaccharide, EDC and buffer ions. The dialyzed preparation was lyophilized to afford the conjugates. The conjugation conditions are summarized in Table 2.

TABLE 2

| Oxidized | Oxidation | Oxidized | | Temperature ° C./ |
|---|---|---|---|---|
| PS^{a} | level (%) | PS (mg) | BSA (mg) | reaction time (hr) |
| Amylose | 66 | 3.1 | 3.1 | 23/48 |
| Nigeran | 15 | 1.2 | 0.6 | 37/72 |

^{a}Polysaccharide

### EXAMPLE 2

### Oxidation of Bacterial Polysaccharide of Actinobacillus and Campylobacter jejuni

After the preliminary work, illustrated in Example 1, the oxidation of bacterial polysaccharides was carried out following the conditions used in Table 1. The first bacterial polysaccharide oxidized was CPS_{Actinobacillus}, a β-(1→6)-glucan (approximately 5500 Da). The results of this study are illustrated in Table 3.

TABLE 3

| | | | | NaClO | | |
|---|---|---|---|---|---|---|
| | | TEMPO | NaBr | (4%) | Reaction | Oxidized |
| PS^{a} | PS (mg) | (mg) | (mg) | (mL) | time (hr) | PS (%) |
| S. suis | 1.93 | 0.05 | 4.0 | 1.0 | 4 | 5 |
| | 5.45 | 0.2 | 3.0 | 0.7 | 8 | 5 |
| C. jejuni | 10.4 | 0.1 | 1.5 | 0.0625 | 20 | 10 |
| | 2.21 | 0.32 | 0.045 | 0.0036 | 8 | 3 |

^{a}Polysaccharide

In the case of CPS_{Actinobacillus}, the backbone of this polysaccharide is resistant to TEMPO oxidation since only the Glc unit at the non-reducing end of the polysaccharide, and a small number of Glc side chains, contain a free primary hydroxyl groups. Subsequently, a slight excess of oxidant was used to ensure that all the terminal Glc units of the CPS_{Actinobacillus} were converted to GlcA residues. Due to the low amount of oxidation in this case, confirmation of oxidation could not be confidently confirmed by 1D ¹H NMR spectroscopy. However, a more sensitive 2D ¹H-¹H NMR HMBC experiment yielded evidence that carboxylic acid moieties had indeed been created, in that a correlation between a proton at 3.69 ppm (H-4 of GlcA and a carboxyl carbon at 174 ppm was observed after oxidation.

As another example, CPS_{Campylobacter} (approximately 6000 Da) from Campylobacter jejuni strain 81176 was oxidized under relatively milder conditions than for Actinobacter. The estimated percentages of oxidized monosaccharide units are illustrated in Table 3. When compared with the 2D ¹H-¹H NMR HMBC spectrum of the intact CPS_{Campylobacter}, a new correlation was observed in the spectrum of the oxidized CPS_{Campylobacter}, between a proton at 3.60 ppm and a carboxyl carbon at 175 ppm after oxidation. A monosaccharide composition analysis designed to detect neutral sugars, showed approximately a 10% decrease of the heptose component, 6dHep, in the oxidized CPS_{Campylobacter} which indicated that oxidation took place mostly at the C-7 position of the 6dHep residue. This is illustrated in FIG. 1. FIG. 1 illustrates the selective oxidation followed by EDC coupling to protein (i.e., BSA). The results are illustrated in Table 4. Corroborating the GC-MS results, the ¹H NMR and ³¹P NMR spectra of the oxidized CPS_{Campylobacter} also showed that the polysaccharide remained structurally intact.

TABLE 4

| Oxidized | Oxidation | Oxidized | | Temperature ° C./ |
|---|---|---|---|---|
| PS^{a} | level (%) | PS (mg) | BSA (mg) | reaction time (hr) |
| CPS_{Actinobacillus} | 5 | 1.85 | 3.7 | 23/4 |
| CPS_{Campylobacter} | 10 | 4.0 | 1.0 | 23/24, 37/48 |

^{a}Polysaccharide

After coupling to BSA, the final conjugate was purified by dialysis and size exclusion column to remove any unconjugated polysaccharides. ¹H NMR experiments on the conjugates showed strong polysaccharide resonances along with some weak BSA signals. Gel electrophoresis revealed the presence of high and low molecular weight conjugates. It is possible that the low molecular weight bands may also contain unconjugated BSA units. More significantly, an immunoblot experiment, using anti-sera against C. jejuni whole cells and anti-sera against CPS_{Campylobacter} conjugated to CRM₁₉₇, recognized the new CPS_{Campylobacter}—BSA conjugate, which indicated that the immunological properties of the CPS_{Campylobacter} in the CPS_{Campylobacter}—BSA conjugate remained intact.

Polysaccharide structures from any number of Campylobacter jejuni strains, including HS3, can be utilized. As such in a preferred embodiment, a method for inducing an anti-Campylobacter jejuni immune response comprising: comprising the steps:
a. Administering the immunogenic composition, where the polysaccharide is conjugated to a protein carrier by the method described above. In this embodiment, immunogenic composition is covalently linked to said protein by reacting said polysaccharide to 2,2,6,6-tetramethylpiperidin-1-oxyl (TEMPO); exposing said TEMPO reacted polysaccharide to an oxidant and exposing said oxidant exposed polypeptide to said protein in the presence of a coupling agent. The oxidant can be any number of compounds, however in one embodiment, NaBr and NaOCl is used, wherein the reaction to The TEMPO is conducted at a pH range of 8.0 to 10.0. In this method the composition is administered at 0.1 μg to 10 mg per dose with or without adjuvant;
b. Administering a boosting dose of said immunogenic composition with or without adjuvant at 0.1 μg to 10 mg per dose.

Any protein conjugate can be used, including CRM₁₉₇. Additionally, the terminal monosaccharide can be other monosaccharrides. However, in a preferred embodiment, the terminal monosaccharide is 6d-ido-Hep and the created carboxylic acid is on residue is C-7. Furthermore, in one embodiment, the polysaccharide contains only 1 to 3 monosaccharide units linked to the protein carrier.

In a preferred embodiment, the polysaccharide polymer is selected from the group consisting of →4)-[P→3]-alpha-D-Gal-(1→3)-[P→2/7]-6d-alpha-D-ido-Hep-(1→; →4)-[P→3]-alpha-D-Gal-(1→3)-[P→2]-L-glycero-alpha-D-ido-Hep-(1→; →3)-6-d-β-D-ido-Hep-(1→4)-β-D-GlcNAc-(1→; and α-D-Gal-(1→[-2)-6d-3-O-Me-α-D-altro-Hep-(1→3)-β-D-GlcNAc-(1→3)-α-D-Gal-]ₙ, wherein is polysaccharide polymer also contains O-methyl-phosphoramide on galactose or heptose, and can also contain 3-hydroxypropanoyl.

### EXAMPLE 3

### Immunogenicity of Campylobacter conjugated to CRM₁₉₇

HS3 capsule polysaccharide contains a heptose monosaccharide it its polysaccharide repeating chain (Aspinall, et al., Eur J. Biochem., 231: 570-578 (1995)). Therefore, attachment to CRM₁₉₇ to the isolated capsule polysaccharide, via the C-7 of 6d-ido-Hep, was undertaken via TEMPO-mediated oxidation followed by EDC-mediated coupling. Illustration of the overall scheme of oxidation and coupling is illustrated in FIG. 2. TEMPO-mediated oxidation was used to avoid disruption of potential immunogenic epitopes of the HS3 CPS. However, in the alkaline condition (pH 10.0, two base-sensitive substitution of O-methyl phosphoramidate and 3-hydroxypropanoyl in the CPS structure were cleaved. This was confirmed by 1D ¹H NMR and illustrated in FIG. 3.

SDS-Polyacrylamide gel electrophoresis (SDS-PAGE) analysis of CPS_{BH-01-0142}-BSA conjugate showed a significant amount of a lower molecular weight band correlating with the presence of CPS_{BH-01-042}-BSA conjugate as a single-ended conjugated. This is shown in FIG. 4. In FIG. 4, lane 1 represents molecular weight markers, lane 2 is CPS_{BH-01-0142}-BSA conjugate and lane 3 is BSA. The molecular weight of BSA was determined to be approximately 67 kDa. However, some degraded BSA was also observed. Additionally, some higher molecular weight conjugate was observed that likely represents cross-linked glycoconjugate.

Anti-sera, raised against whole cells of C. jejuni BH-01-0142 (HS:3,13,50) were reacted to immunoblots of CPS_{BH-01-0142}-BSA conjugate or whole cells of C. jejuni TGH9011 (HS:3 type strain). In this experiment, immune serum reacted to both CPS_{BH-01-0142}-BSA conjugate, as well as to C. jejuni whole cell lysates.

TEMPO-mediated oxidation of CPS polysaccharide was also conducted using the same conditions as for FIG. 2, but with a reduced pH in order to avoid damaging the two non-stoichiometric substitutions of O-methyl phosphoramidate and 3-hydroxypropanoyl groups. In this study the pH value was adjusted to 8.0. Also, in these studies, two quantities of catalytic amounts of oxidant, TEMPO and NaOCl, were used to ascertain the efficiency of oxidation. The structural integrity of CPS_{BH-01-0142} was subsequently confirmed by gas liquid chromatography, mass spectroscopy (GLC-MS) (FIG. 5), and by ¹H NMR and ³¹P NMR (FIG. 6). In FIG. 5, panels (A) and (B) represent intact CPS and activated CPS of C. jejuni BH-01-0142 by TEMPO oxidation at pH 8.0, respectively. In FIG. 6, panels (A) and (B) represent the results of 1D ¹H NMR spectrum and ³¹P NMR spectrum, respectfully, of C. jejuni BH-01-0142 of the activated CPS by TEMPO-mediated oxidation at pH 8.0.

The GLC-MS of the alditol acetate derivatives, of the TEMPO mediated oxidation at pH 8.0, revealed the decreasing traces of 6d-ido-Hep (2.4%) while the other two monosaccharides, i.e., Gal and LD-ido-Hep, remained at a similar amount to that in CPS prior to oxidation. Therefore, the data suggests that the oxidation site was mainly at the C-7 of the α-6d-ido-Hep containing the primary alcohol group, which is selectively oxidized by TEMPO-mediated oxidation, compared with the other sterically hindered secondary alcohol groups in the polysaccharide chain.

SDS-PAGE analysis of CPS_{BH-01-0142}-CRM₁₉₇ (Anderson, Infection and Immunity, 39: 233-238 (1983) conjugate, via TEMPO-mediated oxidation followed by EDC coupling was conducted. The results are illustrated in FIG. 7. In FIG. 7 (A), lane 1 are molecular weight markers, land 2 is CRM₁₉₇, and lane 3 is CPS_{BH-01-0142}-CRM₁₉₇ conjugate. As shown in FIG. 7(A), both lower and higher molecular weight bands, which suggest the presence of single-ended and lattice-type conjugates, respectively.

FIG. 7(B) shows an immunoblot using antisera raised against whole cell C. jejuni BH-01-0142 (HS:3, 13, 50). As seen in panel (B), the antisera recognized the TEMPO-derived CPS_{BH-01-0142}-CRM₁₉₇ glycoconjuate, in which two substitutions of MeOPN and 3-hydroxypropanoyl remained intact.

Evaluation of TEMPO conjugated anti-Campylobacter polysaccharide conjugates were evaluated for immunogenicity in mice. In this study HS:3 CPS_{BH-0142}-CRM₁₉₇ conjugate were used to immunize, subcutaneously, BALB/c mice in aluminum hydroxide three (3) times, at four (4) week intervals. Serum was then collected two weeks following each immunization. Capsule specific IgG responses were then determined by ELISA. The results of this study are illustrated in FIG. 8. were then conjugated to CRM₁₉₇. In FIG. 8, the data represent the mean (+/−)SEM) reciprocal IgG endpoint titer per treatment group. As illustrated in FIG. 8, the CPS conjugate induced a significant level of IgG titer over PBS alone, with a p,0.05 as determined by Tukey's Multiple Comparisons Test.

Obviously, many modifications and variations of the present invention are possible in light of the above teachings. It is therefore to be understood that, within the scope of the appended claims, the invention may be practiced otherwise than as specifically described.

1. An immunogenic composition, composed of a repeating Campylobacter jejuni capsule polysaccharide polymer from one or more Campylobacter jejuni strains, wherein said polysaccharide polymer is covalently linked to a carrier protein via carboxylic acid residues at primary hydroxyl sites.

2. The immunogenic composition of claim 1, wherein said monosaccharide is 6d-ido-Hep and wherein said carboxylic acid residue is primarily at C-7.

3. The immunogenic composition of claim 1, wherein said protein is CRM₁₉₇.

4. The immunogenic composition of claim 1, wherein said polysaccharide polymer is selected from the group consisting of →4)-[P→3]-alpha-D-Gal-(1→3)-[P→2/7]-6d-alpha-D-ido-Hep-(1→; →4)-[P→3]-alpha-D-Gal-(1→3)-[P>2]-L-glycero-alpha-D-ido-Hep-(1→; →3)-6-d-β-D-ido-Hep-(1→4)-β-D-GlcNAc-(1→; and α-D-Gal-(1→[-2)-6d-3-O-Me-α-D-altro-Hep-(1→3)-β-D-GlcNAc-(1→3)-α-D-Gal-]ₙ.

5. The immunogenic composition of claim 1, wherein said polysaccharide polymer also contains O-methyl-phosphoramide on galactose or heptose.

6. The immunogenic composition of claim 1, wherein said polysaccharide polymer contains 3-hydroxypropanoyl.

7. The immunogenic composition of claim 1, wherein said polysaccharide polymer is derived from the HS3 strain of Campylobacter jejuni.

8. The immunogenic composition of claim 1, wherein said polysaccharide contains only 1 to 3 monosaccharide units are linked to said protein carrier.

9. A method of conjugating a bacterial capsule polysaccharide[s], containing a primary alcohol, comprising the steps:
a. reacting a bacterial capsule polysaccharide, containing a primary alcohol, with 2,2,6,6-tetramethylpiperidin-1-oxyl (TEMPO) to create a carboxylic acid;
b. exposing TEMPO reacted polysaccharide of step (a) to TEMPO and oxidant;
c. exposing oxidized polysaccharide of step (b) to a protein carrier in the presence of a coupling agent.

10. The method of claim 9, wherein the oxidant is NaBr and/or NaOCl.

11. The method of claim 9, wherein said oxidant is exposed to NaOCl at a range of 0.69 to 1.4% .

12. The method of claim 9, wherein said coupling agent is 1-ethyl-3-(3-dimethylaminopropyl)carbodiimide.

13. The method of claim 9, wherein said steps reacting to said TEMPO is conducted at a pH range of 8.0 to 10.0.

14. The method of claim 9, wherein said step (c) is conducted at a temperature of 23° C. to 37° C.

15. The method of claim 9, wherein said primary alcohol is contained on the monosaccharide is 6d-ido-Hep and wherein said carboxylic acid residue is primarily at C-7.

16. A method of inducing anti-Campylobacter jejuni immunity comprising the steps:
a. Administering the immunogenic composition of claim 1 at 0.1 μg to 10 mg per dose with or without adjuvant;
b. Administering a boosting dose of said immunogenic composition with or without adjuvant at 0.1 μg to 10 mg per dose.

17. The method of claim 16, wherein said terminal monosaccharide of said immunogenic composition is 6d-ido-Hep and wherein said carboxylic acid residue is C-7.

18. The method of claim 16, wherein said immunogenic composition of claim 1 is covalently linked to said protein by reacting said polysaccharide to 2,2,6,6-tetramethylpiperidin-1-oxyl (TEMPO); exposing said TEMPO reacted polysaccharide to an oxidant and exposing said oxidant exposed polypeptide to said protein in the presence of a coupling agent.

19. The method of claim 16, wherein said protein is CRM₁₉₇.

20. The method of claim 16, wherein is polysaccharide polymer also contains O-methyl-phosphoramide on galactose or heptose.

21. The method of claim 16, wherein said polysaccharide polymer is selected from the group consisting of →4)-[P→3]-alpha-D-Gal-(1→3)-[P→2/7]-6d-alpha-D-ido-Hep-(1→; →4)-[P→3]-alpha-D-Gal-(1→3)-[P→2]-L-glycero-alpha-D-ido-Hep-(1→; →3)-6-d-β-D-ido-Hep-(1→4)-β-D-GlcNAc-(1→; and α-D-Gal-(1→[-2)-6d -3-O-Me-α-D-altro-Hep-(1→3)-β-D-GlcNAc-(1→3)-α-D-Gal-]ₙ.

22. The method of claim 16, wherein said polysaccharide polymer is derived from the HS3 strain of Campylobacter jejuni.

23. The method of claim 16, wherein said polysaccharide polymer contains 3-hydroxypropanoyl.

24. The method of claim 16, wherein said polysaccharide contains only 1 to 3 monosaccharide units linked to said protein carrier.

25. The method of claim 16, wherein said oxidant is NaBr and NaOCl and wherein said reacting to TEMPO is conducted at a pH range of 8.0 to 10.0.

26. The method of conjugating a bacterial capsule polysaccharide, wherein said bacterial capsule polysaccharide is a repeating Campylobacter jejuni polysaccharide polymer from one or more Campylobacter jejuni strains.

27. The method of claim 26, wherein said Campylobacter jejuni polysaccharide polymer from one or more Campylobacter jejuni strains is selected from the group consisting of →4)-[P→3]-alpha-D-Gal-(1→3)-[P→2/7]-6d-alpha-D-ido-Hep-(1→; →4)-[P→3]-alpha-D-Gal-(1→3)-[P→2]-L-glycero-alpha-D-ido-Hep-(1→; →3)-6-d-β-D-ido-Hep-(1→4)-β-D-GlcNAc-(1→; and α-D-Gal-(1→[-2)-6d-3-O-Me-α-D-altro-Hep-(1→3)-β-D-GlcNAc-(1→3)-α-D-Gal-]ₙ.
